(19) United States
(12) Patent Application Publication
(10) Pub. No.: US 2008/0255534 A1
(43) Pub. Date: Oct. 16, 2008

(54) Disposable article with novel refastenable system

(75) Inventor: Loida Guzman Reyes, Philadelphia, PA (US)

Correspondence Address: AMSTER, ROTHSTEIN & EBENSTEIN LLP, 90 PARK AVENUE, NEW YORK, NY, 10016, US

(73) Assignee: Tyco Healthcare Retail Services, AG, Schaffhausen (CH)

(21) Appl. No.: 11/787,038

(22) Filed: Apr. 12, 2007

(51) Int. Cl. A61F 13/62

(52) U.S. Cl. 604/391

(57) ABSTRACT

A disposable absorbent article has an absorbent chassis, two independent side panels, a pair of front releasable fastening systems and a pair of back releasable fastening systems. Each side panel comprises a pair of opposite transverse edges. The front fastening system releasably attaches the front waist region to a transverse edge of the side panel. The back fastening system releasably attaches the back waist region to an opposite transverse edge of the side panel.

### BACKGROUND OF THE INVENTION

The present invention relates to disposable, absorbent pant-like articles for containing body exudates. More specifically, the present invention relates to an absorbent article with independent front releasable fastening systems and back releasable fastening systems.

Conventional disposable absorbent pant-like articles have a structure that comprises an absorbent chassis and one or more side panels. The absorbent chassis comprises an outer liner, a body liner and an absorbent core disposed between the two liners. The absorbent chassis defines a front waist region, a back waist region and a crotch region extending between and connecting the front and back waist regions. The absorbent chassis generally has two laterally extending side panels that are permanently fixed to its back waist region. Both the elastic side panels can be fastened, unfastened and refastened to the front waist region of the absorbent chassis.

Conventional absorbent articles are desired to have the best possible fit for wearers. At the same time, these absorbent articles should be convenient for caregivers or nurses to put on and take off wearers. The convenience pertains to the ease with which the caregivers or nurses put on or take off the absorbent article without soiling or contaminating the wearer and without changing the position of the wearer.

Generally, in conventional absorbent articles, the transverse edges of the two waist regions are connected by one or more side panels through only one seam or releasable fastening system at each side waist region. Consequently, the seam could be either at the front waist region, at the back waist region or at the side waist region. Thus, for a wearer lying face down (or face up) in a bed wearing a conventional absorbent article such as a diaper, the fastening seams are typically in the front waist regions (or back waist region), and it would be difficult to take the diaper off without tearing away the side panels or without changing the position of the wearer. Moreover, for bedridden wearers lying face down or face up or sideways, there is need for a type of disposable article that could be taken off without changing the position of the wearer.

Further, in conventional disposable articles, the side panels are permanently affixed to the front waist region or back waist region of the chassis using typical sealing techniques like ultrasonic bonding, thermal bonding, chemical bonding, or other techniques which make the edges of the garment rough with a poor appearance. The rough edges resulting from such treatment could cause potential irritation to the skin and account for the poor appearance of the absorbent articles.

Therefore, there exists a need for overcoming the disadvantages generally associated with disposable articles formed using treatments like ultrasonic bonding, thermal bonding, chemical bonding and the like.

There also exists a need for a new absorbent article that allows wearers or caregivers to more conveniently wear or take off the absorbent article from the wearer, in various wearer positions.

### SUMMARY

In response to the above needs, a new disposable absorbent article is provided. According to an aspect of the invention, the absorbent article can desirably be fastened to provide a pant-like appearance and is convenient for both wearers to wear and nurses and caregivers to put on and take off. The absorbent article has multiple independent releasable fastening systems, placed at both the front waist region and back waist region of the article. Each fastening system can be fastened, unfastened and refastened independently. The independent operation of each fastening system allows nurses and caregivers to take the pant-like article off the wearer without changing the position of the wearer and without tearing the side panels of the pant-like article.

In another aspect, the absorbent article comprises an absorbent chassis, a pair of independent side panels, a pair of front releasable fastening systems, and a pair of back releasable fastening systems. The absorbent chassis defines a longitudinal axis, a transverse axis perpendicular to the longitudinal axis, a pair of opposite front transverse edges, and a pair of opposite back transverse edges. Further, the absorbent chassis is divided into a front waist region, a back waist region longitudinally separated from the front waist region, and a crotch region that extends between and connects the front waist region and the back waist region.

Each of the side panels defines opposite longitudinal edges and opposite transverse edges. The side panel and fastening systems releasably connect the front transverse edge and the back transverse edge of the absorbent chassis.

Each back releasable fastening system comprises one fastening component attached to the back waist region disposed in the back transverse edge and one mating component attached to the side panel disposed in one transverse edge of the side panel. Two fastening components from each back transverse edge releasably engage two mating components from the two side panels.

Similarly, each front releasable fastening system comprises one fastening component attached to the front waist region disposed in the front transverse edge and one mating component attached to the side panel disposed in the opposite transverse edge. Two fastening components from each of the opposite front transverse edge releasably engage two mating components from the opposite transverse edges of the two side panels.

Two front and two back releasable fastening systems secure the front and back waist regions together with the side panels around the waist. If the absorbent article becomes soiled during use and the wearer is lying facing down in the bed, then the absorbent article can be taken off easily by unfastening the back releasable fastening systems without changing the position of the wearer. Further, if the wearer is lying facing up in the bed, the pant-like article can be taken off easily by unfastening the front releasable fastening systems without changing the position of the wearer. Thus the absorbent article is configured to be pulled on and taken off the wearer by engaging and disengaging independent front and back releasable fastening systems. This can be done without changing the position of the wearer and without tearing away the side panels of the absorbent article. Unlike the conventional absorbent articles, any person can fasten, unfasten or refasten the absorbent article from either front or back without changing the position of the wearer.

The absorbent articles provided by the present invention are adapted to be worn adjacent to the body of the wearer and contain various bodily fluids. The absorbent articles can desirably be fastened to provide pant-like articles for the user. This pant-like article can be pulled on like a conventional pant, subsequently checked for soiling and removed with an added ease compared to conventional pant-like articles. Moreover, the absorbent article can be used like a diaper rather than like a pant.

Further, the absorbent article has better appearance and substantially prevents irritation to the skin as rough edges are rarely present in the absorbent article since the side panels are independent of the chassis.

### DRAWINGS

The foregoing and other advantages and features of the invention will become apparent upon reading the following detailed description and upon reference to the drawings in which:

FIG. 1 illustrates a plan view of an absorbent article according to an embodiment of the invention;

FIG. 2 illustrates a plan view of the absorbent article of FIG. 1 in a configuration in which the transverse edges of the side panels have been fastened to the transverse edges of the back waist region;

FIG. 3 illustrates a schematic view of the absorbent article of FIG. 2, the article being folded about the crotch region;

FIG. 4 illustrates a front perspective view of an absorbent article in a pant-like shape in accordance with an embodiment of the present invention;

FIG. 5 illustrates side panels with curved longitudinal edges in accordance with an embodiment of the invention; and

FIG. 6 illustrates side panels with elastic portions in accordance with another embodiment of the invention.

### DETAILED DESCRIPTION

As used herein, the terms “absorbent article”, or simply “article”, refer to garments that absorb and contain body fluids and other body exudates. More specifically, these terms refer to garments that are placed against or in proximity to the body of a wearer to absorb and contain the various exudates discharged from the body. A non-exhaustive list of examples of such absorbent garments includes feminine hygiene products, diapers, disposable diapers, training pants, and adult incontinence products. Such garments may be intended to be discarded or partially discarded after a single use (“disposable” garments). Such garments may comprise essentially a single inseparable structure (“unitary” garments), or they may comprise replaceable inserts or other interchangeable parts.

The present invention may be used with all of the foregoing classes of absorbent articles, without limitation, whether disposable or otherwise. The absorbent article of the present invention is optionally a diaper, feminine hygienic garment, incontinent brief, training pant, diaper, or combinations thereof.

In the foregoing specification, specific embodiments of the present invention have been described. However, one having ordinary skill in the art will appreciate that various modifications and changes can be made without departing from the spirit and scope of the present invention as set forth in the claims below. Accordingly, the specification and figures are to be regarded in an illustrative rather than a restrictive sense, and all such modifications are intended to be included within the scope of the present invention. The benefits, advantages, solutions to problems, and any element(s) that may cause any benefit, advantage, or solution to occur or become more pronounced are not to be construed as critical, required, or essential features or elements of any or all the claims.

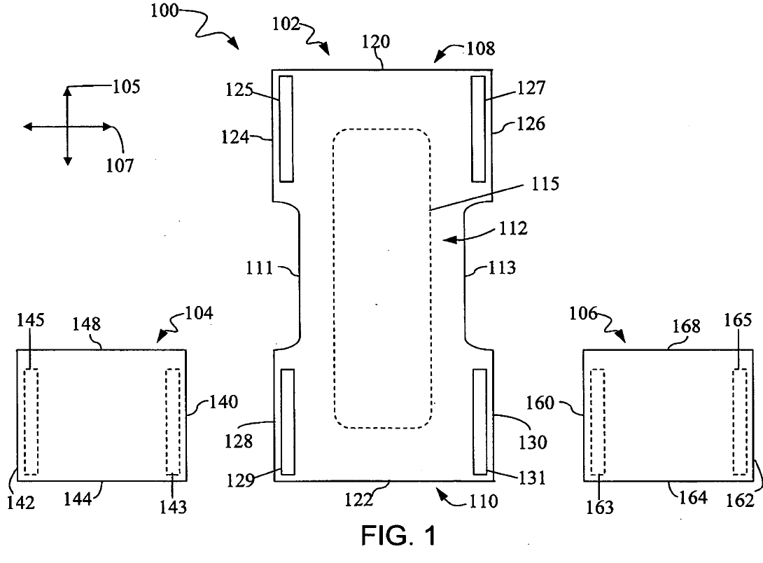

In FIG. 1, an absorbent article 100 is illustrated in an unfastened and laid flat condition. Article 100 comprises an absorbent chassis 102 and two side panels 104 and 106. Absorbent chassis 102, defines a longitudinal axis 105, a transverse axis 107 perpendicular to the longitudinal axis, a front waist region 108, a back waist region 110 longitudinally separated from front waist region 108, and a crotch region 112 interconnecting the two waist regions. Absorbent chassis 102 comprises an outer liner, a body liner, and an absorbent core 115 placed between the outer liner and the body liner.

Side panels 104 and 106 are independent of absorbent chassis 102 and can releasably attach to the opposite waist regions 108, 110. Side panel 104 has two opposite transverse edges 140 and 142, and two opposite longitudinal edges 144 and 148. Similarly, side panel 106 has two opposite transverse edges 160 and 162, and two opposite longitudinal edges 164 and 168.

The absorbent chassis further defines longitudinally opposite waist edges 120 and 122, a pair of opposite front transverse edges 124 and 126 of the front waist region 108, and another pair of opposite back transverse edges 128 and 130 of the back waist region 110. Crotch region 112 comprises opposite crotch transverse edges 111 and 113.

In FIG. 1, four identical fastening components 125, 127, 129 and 131 are attached to chassis 102. Two mating components 125 and 127 are disposed in front transverse edges 124 and 126 of the front waist region 108, and other two fastening components 129 and 131 are disposed in back transverse edges 128 and 130 of the back waist region 110 of the chassis. Corresponding to the fastening components, there are four mating components 143, 145, 163 and 165. Two mating components 143 and 145 are disposed on opposite transverse edges 140 and 142 of the side panel 104, and similarly two other mating components 163 and 165 are disposed on opposite transverse edges 160 and 162 of side panel 106. It can be appreciated here that although identical fastening or mating components are illustrated in this aspect of the invention, the chassis 102 may comprise at least one different type of fastening component. Moreover, fastening components and mating components other than those illustrated herein will be apparent to those skilled in art, and may be used interchangeably in various embodiments of the present invention.

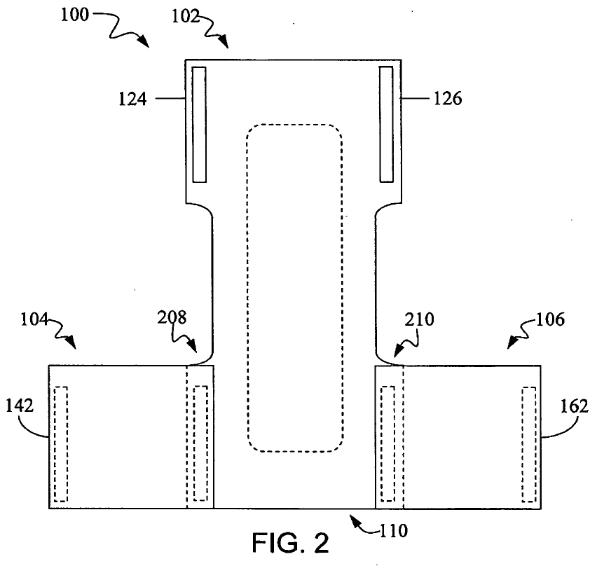

A fastening component is designed to releasably engage the mating component, thereby forming a releasable fastening system. FIG. 2 shows two back releasable fastening systems 208 and 210 disposed in back waist region 110 of chassis 102. Fastening system 208 comprises fastening component 129 (shown in FIG. 1) releasably engaging mating component 143 (shown in FIG. 1), as shown by the dotted rectangle in FIG. 2. Similarly, fastening system 210 comprises a fastening component 131 (shown in FIG. 1) releasably engaging mating component 163 (shown in FIG. 1), as shown by the dotted rectangle.

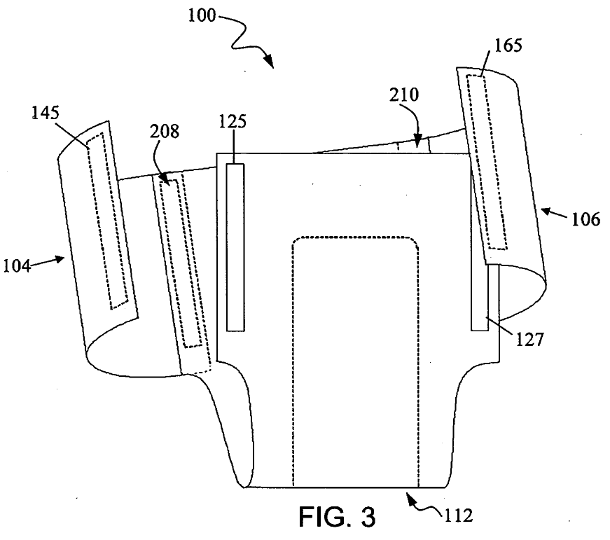

As shown in FIG. 2, back fastening systems 208 and 210 releasably engage side panels 104 and 106 to the back waist region 110 of chassis 102 forming an inverted T shape. Chassis 102 along with attached side panels 104 and 106 can be folded about crotch region 112 as shown in FIG. 3. Mating components 145 and 165 then become available to engage fastening components 125 and 127.

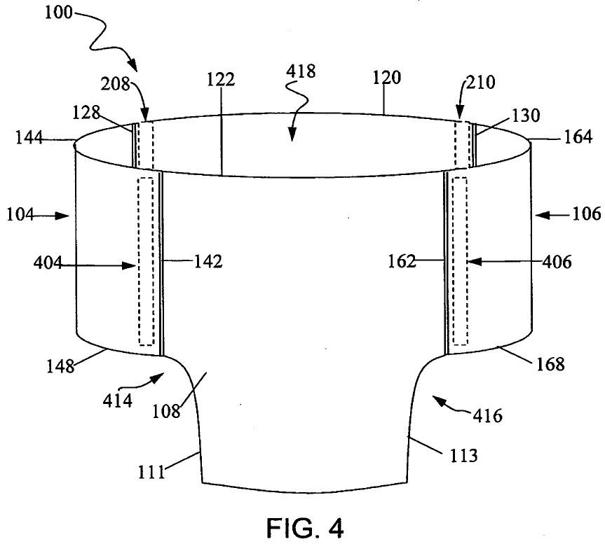

The fastening component 125 releasably engages mating component 145 constituting a front releasable fastening system 404 as shown in FIG. 4 by the dotted rectangle. Similarly, fastening component 127 releasably engages mating component 165 constituting another front releasable fastening system 406 as shown in FIG. 4 by the dotted rectangle.

FIG. 4 shows an embodiment of a pant-like absorbent article according to the present invention. The absorbent article comprises two front releasable fastening systems 404 and 406, and two back releasable fastening systems 208 and 210. Front releasable fastening systems 404 and 406 releasably engage side panels 104 and 106 with front waist region 108 of the chassis. Similarly, back releasable fastening systems 208 and 210 are disposed in back waist region 110 and releasably engage side panels 104 and 106 with the back waist region of the chassis. The two back releasable fastening systems are shown in FIG. 4 by a pair of dotted rectangles marked 208, 210. In the embodiment illustrated in FIG. 4, all of the transverse edges of the two side panels are configured to be placed onto the transverse edges of the chassis. Consequently, all the transverse edges of the side panels are visible from the outside of the garment. In alternate embodiments, at least one transverse edge of the chassis is configured to be placed onto the transverse edges of at least one of the side panels 104 and 106, and consequently, the at lest one transverse edge of the chassis is visible from the outside.

In FIG. 4, transverse edge 111 of the crotch region together with longitudinal edge 148 form one leg opening 414, and similarly, transverse edge 113 of the crotch region together with longitudinal edge 168 form another leg opening 416. Longitudinal waist edges 120 and 122 of the absorbent chassis, together with longitudinal edges 144 and 164 of side panel 104 and 106, constitute a waist opening 418. Transverse edges 142 and 162 are illustrated in front waist region 108 near the two fastenable systems 404 and 406 shown in FIG. 4. Similarly, parts of each of transverse edges 128 and 130 are illustrated in the back waist region near the two fastenable systems 208 and 210 in FIG. 4.

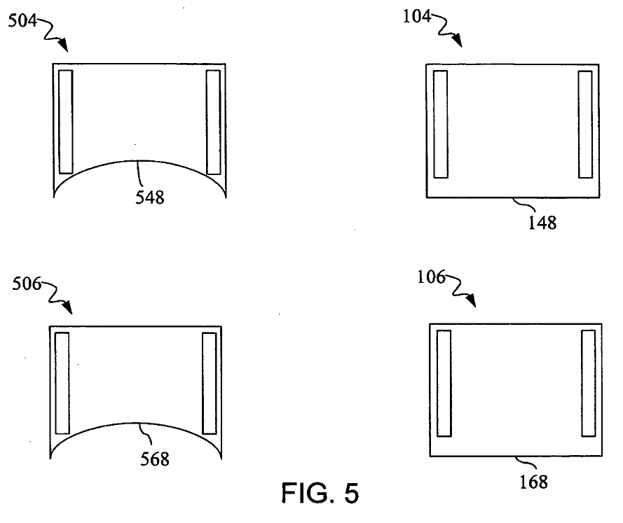

FIG. 5 illustrates another aspect of the present invention where the side panels have curved longitudinal edges. Each side panel 104 and 106 has two longitudinal edges. Longitudinal edge 148 of side panel 104 and longitudinal edge 168 of side panel 106 constitutes the two leg openings as discussed before. In this particular embodiment, side panels 504 and 506 have curved longitudinal edges 548 and 568 as in FIG. 5 for best fit and good appearance.

Absorbent chassis 102 of the present absorbent article 100 may have various different profiles/shapes. In various embodiments, chassis 102 has a rectangular or a T-shape profile. As discussed above, the absorbent chassis 102 comprises an outer liner, a body liner and an absorbent core disposed between the two layers. Typically, the body liner is a water-pervious layer, and the outer liner is a water impervious layer. The body liner is formed from a material such as a fibrous non-woven fabric or porous plastic film. Preferred non-woven materials include spun-bonded polypropylene, spunbonded polyethylene, and thermally bonded webs of staple fibers. The outer liner is made from material such as plastic film or a laminate sheet of plastic film. In one embodiment, white AVGOL/Pantex forms the outer liner. In different alternate embodiments, where the absorbent article may be used as a diaper or a training pant for children, the outer liner of the chassis may have various film designs.

The absorbent core may be made up of any suitable absorbent material, as well as combinations of different types of absorbent materials. For example, the absorbent core may be formed from a mixture of pulp fluff and superabsorbent particles wrapped in a liquid permeable tissue wrap. The superabsorbent particles may be substantially homogeneously mixed with the hydrophilic fibers, or they may be non-uniformly mixed. The pulp fluff may be exchanged with synthetic, polymeric, meltblown fibers, or with a combination of meltblown fibers and natural fibers. Examples of superabsorbent materials include polyacrylamides, polyvinyl alcohol, polyacrylates, various grafted starches, and the like.

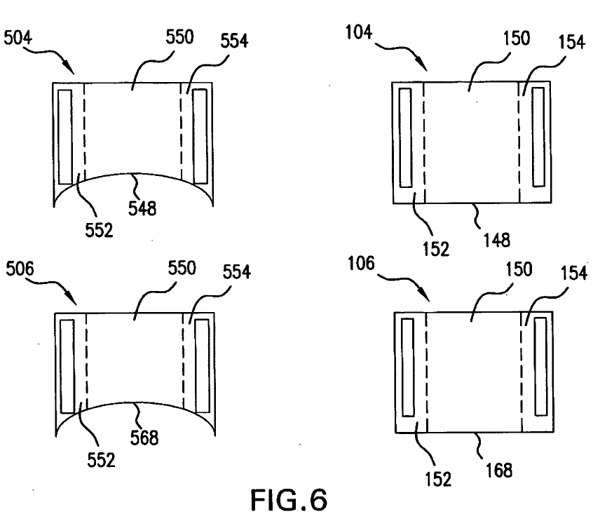

Referring now to FIG. 6, side panels 104 and 106, or side panels 504 and 506, are preferably constructed of an elastic material capable of stretching in transverse axis direction 107. In an alternative embodiment, side panels 104, 106 and/or 504, 506 can be formed as a three-piece construction having a central panel 150, 550 which is made from a non-elastic material, and transverse edge sections 152, 154 and 552, 554 on either side of the central panel 150, 550 which are made from an elastic material. In this alternative embodiment, the elastic transverse edge sections 152, 154 and 552, 554 can include the respective mating components attached thereto. It will further be understood that one of the mating pairs of elastic transverse edge sections 152, 154 and 552, 554, for example transverse edge section 152 and/or 552 could be made from an elastic material and the other of the mating pairs, such as 154 and/or 554, could be made from a non-elastic material.

In a further alternative embodiment of the three-piece construction side panels 104, 106 and/or 504, 506, the central panel 150, 550 can instead be made from an elastic material, and the transverse edge sections 152, 154 and 552, 554 can be made from a non-elastic material. In this alternative embodiment, the non-elastic transverse edge sections 152, 154 and 552, 554 can include the respective mating components attached thereto.

The side panels 104, 106 and 504, 506 of the absorbent articles may desirably be constructed of an elastic material capable of stretching in the transverse axis direction. For example, the side panels 104, 106 and 504, 506 may comprise a stretch-thermal laminate (STL), neck-bonded laminate (NBL), or stretch-bonded laminate (SBL) material. In some embodiments of the present invention, each of the side panels 104, 106 and 504, 506 may be formed from a less stretchable film-pliant material such as Blue-GPF. In various other embodiments of the present invention, side panels 104 and 504 may be different from side panels 106 and 506. Side panels 104 and 504 may be formed of different types of materials than the materials that are used in the construction of side panels 106 and 506.

The fastening components attached to the chassis 102, and the mating components attached to side panels 104 and 106, can be of various types including hooks, loops, hook to loop, snap fastener, adhesive, other mechanical fasteners and the like. In certain embodiments, the absorbent articles comprise hook to hook or hook to loop fastening components. One skilled in the art can recognize that the shape, size and number density of the hooks and loops can be varied to obtain the desired level of securement between the fastening components and mating components.

In various other embodiments of the present invention, the fastening components and the mating components may be used interchangeably. In various other embodiments of the present invention, at least one of the fastening components may comprise a plurality of rows of fastening components. At least one of the plurality of rows of fastening components comprises at least one gap between at least two fastening components. Corresponding to the design of the fastening components, at least one of the side panels comprises a plurality of rows of mating components. At least one of the plurality of rows of mating components comprises at least one other gap between at least two mating components. The gap allows an easy bending of the fastening components and the mating components in conformity with the movement of the wearer's body. The gap may also prevent popping up of the side panels from the chassis caused by disengaging the mating components from fastening components. For example, a row of buttons, for example about 2 to 4, may be received in each of the side panels. Corresponding to the row of buttons, multiple slots are created on the chassis for receiving the buttons. The row of buttons and slots may be placed interchangeably on the chassis and the side panels. Each of the buttons is placed into one of the slots engaging each of the side panels to the chassis.

As will be apparent to those skilled in the art, the various aspects of an absorbent article as discussed above exhibit many advantages over conventional methods. The absorbent article is convenient for both wearers to wear and nurses and caregivers to put on and take off the wearer. The absorbent article has a plurality of independent releasable fastening systems. The independent operation of each fastening system allows nurses and caregivers to take the pant-like article off the wearer without changing the position of the wearer and without tearing the side panels of the pant-like article. The absorbent articles can desirably be fastened to provide pant-like articles for the user. This pant-like article can be pulled on like a conventional pant, subsequently checked for soiling and removed with added ease compared to conventional pant-like articles. Advantageously, a wearer can fasten, unfasten, subsequently check for soiling and refasten the absorbent article from either the front or back without changing the position of the wearer. Furthermore, the absorbent article has a better appearance and substantially prevents irritation to the skin as rough edges are rarely present in the absorbent article since the side panels are independent of chassis.

The aforementioned embodiments and examples are meant to be solely for explanatory purposes, and are not meant to be limiting to any of the embodiments presented herein. Various other configurations, modifications or variations will be apparent to those skilled in the art. Such configurations are within the spirit of the invention, and are included within the boundaries defined by the appended claims.

What is claimed is:

1. A disposable absorbent article comprising:
an absorbent chassis defining a longitudinal axis and a transverse axis perpendicular to the longitudinal axis, wherein the absorbent chassis is divided into
a front waist region having a first front transverse edge and an opposed second front transverse edge,
a back waist region having a first back transverse edge and an opposed second back transverse edge, and
a crotch region that extends between and connects the front waist region and the back waist region;
a first side panel releasably connecting the first front transverse edge and the first back transverse edge; and
a second side panel releasably connecting the second front transverse edge and the second back transverse edge.

2. The absorbent article as claimed in claim 1, wherein the first side panel defines a first side panel transverse edge and a second side panel transverse edge such that the first side panel transverse edge is releasably connected to the first front transverse edge and the second side panel transverse edge is releasably connected to the first back transverse edge; and
wherein the second side panel defines a third side panel transverse edge and a fourth side panel transverse edge such that the third side panel transverse edge is releasably connected to the second front transverse edge and the fourth side panel transverse edge is releasably connected to the second back transverse edge.

3. The absorbent article as claimed in claim 2, further comprising a first fastening component disposed on the first front transverse edge, a second fastening component disposed on the first back transverse edge, a third fastening component disposed on the second front transverse edge and a fourth fastening component disposed on the second back transverse edge.

4. The absorbent article as claimed in claim 3, wherein the first side panel transverse edge includes a first mating component releasably attached to the first fastening component, the second side panel transverse edge includes a second mating component releasably attached to the second fastening component, the third side panel transverse edge includes a third mating component releasably attached to the third fastening component, and the fourth side panel transverse edge includes a fourth mating component releasably attached to the fourth fastening component.

5. The absorbent article as claimed in claim 1, further comprising a first fastening component disposed on the first front transverse edge, a second fastening component disposed on the first back transverse edge, a third fastening component disposed on the second front transverse edge and a fourth fastening component disposed on the second back transverse edge.

6. A disposable absorbent article comprising:
an absorbent chassis defining a longitudinal axis and a transverse axis perpendicular to the longitudinal axis, the absorbent chassis further defining a pair of opposed front transverse edges and a pair of opposed back transverse edges, wherein the absorbent chassis comprises
a front waist region,
a back waist region longitudinally separated from the front waist region, and
a crotch region that extends between and connects the front waist region and the back waist region;
at least one side panel releasably connecting the front transverse edge and the back transverse edge of the absorbent chassis, the at least one side panel defining a pair of opposed longitudinal side panel edges and a pair of opposed transverse side panel edges;
at least one front releasable fastening system comprising at least one fastening component attached to the front waist region, the at least one fastening component disposed on the front transverse edge, and at least one mating component attached to the at least one side panel, the at least one mating component disposed on the transverse side panel edge of the at least one side panel, the at least one fastening component releasably engaging the at least one mating component; and
at least one back releasable fastening system comprising at least one fastening component attached to the back waist region, the at least one fastening component disposed on the back transverse edge, and at least one mating component attached to the at least one side panel disposed on the transverse side panel edge of the at least one side panel, the at least one fastening component releasably engaging the opposite at least one mating component.

7. The absorbent article as claimed in claim 6, wherein the absorbent article is a pant-like article comprising two side panels, a pair of front releasable fastening systems and a pair of back releasable fastening systems.

8. The absorbent article of claim 6, wherein the at least one side panel is elastic.

9. The absorbent article of claim 6, wherein the at least one side panel has at least one curved longitudinal edge.

10. The absorbent article of claim 6, wherein the at least one fastening component and the at least one mating component of the at least one releasable fastening system are hook to hook.

11. The absorbent article of claim 6, wherein the at least one fastening component and the at least one mating component of the at least one releasable fastening system are hook to loop.

12. The absorbent article of claim 6, wherein the at least one fastening component and the at least one mating component of the at least one releasable fastening system is a button and a slot.

13. The absorbent article of claim 6, wherein the at least one fastening component and the at least one mating component of the at least one releasable fastening system are bonded by an adhesive.

14. The absorbent article of claim 6, wherein the at least one mating component disposed on the at least one side panel is attached to the at least one fastening component disposed on the chassis.

15. The absorbent article of claim 6, wherein the at least one fastening component of the chassis is attached to the at least one mating component disposed on the at least one side panel.

16. The absorbent article of claim 6, wherein the at least one fastening component comprises a plurality of fastening components arranged in at least one row of fastening components, and the at least one mating component comprises a plurality of mating components arranged in at least one row of mating components.

17. A diaper comprising:
an absorbent chassis comprising an outer liner sheet, a body liner sheet and an absorbent core placed between the two sheets; the absorbent chassis defining a longitudinal axis, a transverse axis perpendicular to the longitudinal axis, the absorbent chassis further defining a pair of opposed front transverse edges and a pair of opposed back transverse edges, wherein the absorbent chassis is divided into
a front waist region,
a back waist region longitudinally separated from the front waist region, and
a crotch region that extends between and connects the front waist region and the back waist region, the crotch region defining two opposite crotch transverse edges;
at least one elastic side panel releasably connecting the front transverse edge and the back transverse edge of the absorbent chassis, the side panel defining opposed transverse edges;
at least one front releasable fastening system comprising at least one fastening component attached to the front waist region disposed in the front transverse edge and at least one mating component attached to the side panel disposed in the transverse edge of the side panel, the fastening component releasably engaging the mating component;
at least one back releasable fastening system comprising at least one fastening component attached to the back waist region disposed in the back transverse edge and at least one mating component attached to the side panel disposed in the opposite transverse edge of the side panel, the fastening component releasably engaging the opposite mating component;
a pair of elastomeric components attached to the crotch region disposed in opposite transverse crotch edges; and
containment flaps enclosing the absorbent core of the absorbent chassis.
